(19) United States
(12) Patent Application Publication
(10) Pub. No.: US 2024/0368522 A1
(43) Pub. Date: Nov. 7, 2024

(54) Method and apparatus for continuous algae production

(71) Applicant: PUEVIT GmbH, Dresden (DE)

(72) Inventors: Gunnar MUEHLSTAEDT, Dresden (DE); Felix KRUJATZ, Dresden (DE)

(73) Assignee: PUEVIT GmbH, Dresden (DE)

(21) Appl. No.: 18/650,376

(22) Filed: Apr. 30, 2024

(30) Foreign Application Priority Data
May 2, 2023 (DE) 10 2023 111 197.3

(51) Int. Cl. C12M 1/34; C12M 1/00; C12M 1/12; C12N 1/12

(52) U.S. Cl. CPC C12M 41/36; C12M 21/02; C12M 23/06; C12M 47/02; C12N 1/12

(57) ABSTRACT

A method for continuous production of algae in a nutrient medium within a reactor while maintaining a constant biomass concentration or turbidity comprises the steps: 1. inoculating of a nutrient medium with algae; 2. mixing until a constant biomass concentration of 0.3 0.8 g/L in the nutrient medium is reached; 3. continuous operation of the algae cultivation in the reactor (1) while maintaining this biomass concentration, comprising the steps of: 3a. removing a portion of the nutrient medium, and 3b. separating the algae thereof, and 3c. adding at least a portion of the nutrient medium removed in step 3a. after algae separation as algae-free, reprocessed nutrient medium, with regulating simultaneously at least one control variable during the continuous operation within the reactor, selected from dilution factor, irradiated light quantity and temperature, in order to maintain the constant biomass concentration.

### CROSS-REFERENCE TO RELATED APPLICATION

This application claims the benefit of German Patent Application DE 10 2023 111 197.3, filed on May 2, 2023, the content of which is incorporated by reference in its entirety.

### TECHNICAL FIELD

The disclosure relates to the continuous production of algae in a nutrient medium within a reactor while maintaining the constant biomass concentration or turbidity, respectively.

### BACKGROUND

Algae can be used in many different ways. The technical production of algae is therefore playing an increasingly important role. Various technological systems are widely used for closed photo bioreactors.

Algae cultivation in batch mode is known, as described, for example, in WO 2009/058621 A1.

Other processes work in continuous operation mode.

In AT 5 171 88 A1, for example, a continuous production in a system with horizontal distribution pipes and vertical rising and falling pipes with a meandering flow is described, in which the algae concentration increases to a very high range of 10-50 g/L. When this high concentration range is reached, algae removal is carried out by means of filtration. A disadvantage of the process is the poor volumetric productivity of the algae due to the very high algae concentration. The filters occasionally become clogged and require maintenance.

EP 2 486 790 A1 describes a tubular reactor for the continuous cultivation of microorganisms with CO₂ feed, whereby the gas phase contains at least 0.2% vol.-% CO₂. The flow speeds are 0.2-0.3 m/s, at most 0.5 m/s in any case. The algae biomass according to the first experiment described increases from 0.1 g/L to 3 g/L in order to obtain as much algae as possible.

WO 2010/002745 A1 describes a flat-plate photo bioreactor that is used to investigate algae growth models with the corresponding prediction capability.

US 2018 112165 A1 describes a microalgae bioreactor with a turbidity sensor, whereby cultivation is carried out in batch mode up to an algae concentration of 1 g/L in order to, subsequently, keep the dilution constant within a range of 30%, for example at 0.5-2 g/L, in turbidostat-mode (corresponds to keeping the turbidity constant) via a turbidity sensor. The algae are separated by filtration. A disadvantage is that the filters can become clogged. Disadvantageously, the determination is strongly dependent on the composition of the nutrient medium, so that the error rate in the in-situ determination of the algae concentration is high. US 2018 112165 A1 suggests that the algae concentration should preferably be set much higher, for example >1.0 g/L.

An alternative to turbidostat operation mode is the so called chemostat operation mode, in which the concentration of nutrients in the nutrient medium is kept constant. However, due to an unmonitored algae concentration, this often results in poor algae growth behaviour despite optimal nutrient concentrations.

WO 2018/037402 A1 discloses a bioreactor for growth of cells and microorganisms with recirculation of the nutrient medium and a turbidity sensor as well as a perforated barrier (membrane) between two chambers of the bioreactor for retaining the cells or microorganisms, respectively, in the upper chamber. The design is complex.

With regard to the purification of the so-called culture broths in algae production, it is known to remove algae in batch mode individually batch by batch, e.g. using centrifuges, and to collect these algae this way in order to subsequently dry them.

In continuous operation, on the other hand, the harvesting or separation of the algae is often described as a “bottleneck” step. For example, Lopez-Guzman et al. (2021) or Visigalli et al. (2021) describe combining the methods of flotation and electrocoagulation. This combination is also known from the field of wastewater treatment in the removal of algae from wastewater, as described, for example, in US 2017/0113957 A1.

Many of the known methods for algae production (both in the topic of upstream, i.e. cultivation, and in the downstream topic, i.e. separation) intend open working areas or have a high risk of contamination for other reasons. The algae obtained are therefore not suitable for consumption or the process is not carried out according to standards (e.g. GMP).

### SUMMARY

The disclosure enables simple and effective algae production on an industrial scale. It makes it possible to obtain as much algae biomass as possible in as short a time as possible with as little liquid volume as possible.

This means, among other things, that the invention should make it possible to maximise the volumetric productivity of the algae. The method and the structure of the apparatus/plant should be simple, in order to enable for a cost-effective algae production also on an industrial scale. This also means that the invention should enable maintenance work to be reduced. The processing times without interruption should therefore be as long as possible.

The algae should be suitable for consumption and therefore free from harmful contaminants.

A method for the continuous production of algae in a nutrient medium within a reactor, comprises the steps of
1. inoculating a nutrient medium with algae,
2. mixing until a constant biomass concentration of 0.3-0.8 g/L in the nutrient medium is reached,
3. continuously operating the algae cultivation in the reactor while maintaining this biomass concentration, comprising the steps of:
   3a. removing a portion of the nutrient medium, and
   3b. separating the algae thereof, and
   3c. adding at least a portion of the nutrient medium removed in step 3a. after algae separation as algae-free, reprocessed nutrient medium,
   (so that steps 3a., 3b. and 3c. also run continuously),
   with regulating simultaneously (in continuous operation, i.e. in step 3. of the method) at least one control variable during the continuous operation within the reactor, selected from dilution factor, irradiated light quantity and temperature, in order to maintain the constant biomass concentration.

The “dilution factor” indicates how often the nutrient medium in the reactor is exchanged per time. This ensures dilution and therefore a reduction in optical density of the algae suspension and therefore also in the biomass concentration.

It is therefore calculated from the portion of the nutrient medium removed in step 3a. per time. Since the reactor filling is kept constant in continuous operation, the removed nutrient medium volume is added again in the same volume in step 3c (at least a portion as nutrient medium freed from algae and possibly mixed with new, fresh nutrient medium). A dilution factor of 0.3-1 per day therefore means that 30% to 100% of the total reactor content is completely replaced within 24 hours.

Regarding the “irradiated light quantity”, the influence of the light on the change in biomass concentration (or in turbidity value) can be determined using mathematical calculations and the supply of light can be adjusted accordingly (automatically or manually). This enables particularly energy-efficient operation.

Also setting the “temperature” as a control variable as a function of the biomass concentration at the time of determination provides a way of maintaining the constant biomass concentration of, according to the invention, 0.3-0.8 g/L in the nutrient medium.

An apparatus for the continuous production of algae in a nutrient medium, comprises:
a reactor,
with a turbidity sensor connected to the reactor for monitoring the turbidity of the nutrient medium within the reactor,
an algae removal unit,
a pipe from the reactor to the algae removal unit and from there back to the reactor, and
as a regulation unit, for regulating a control variable inside the reactor,
a pump for regulating a dilution factor, and/or
a light source for regulating the light quantity (which can be measured using a PAR sensor, for example), and/or
a heating/cooling unit for regulating the temperature,
each with a control unit for synchronising the turbidity sensor with the regulation unit so that a turbidity value can be maintained as a setpoint.

Sensibly, an automatically controlled or manually operated feed unit for fresh nutrient medium is comprised.

The apparatus according to the disclosure may be used for the continuous production of algae with the method according to the disclosure.

The conversion of biomass concentration (in g/L) into optical density (at 750 nm) for algae is perfomed using the factor 1/0.86. This means that a biomass concentration of 0.3 g/L corresponds to an optical density of 0.35, for example.

The skilled person recognises which steps of the method according to the invention are carried out with which components of the apparature. Accordingly, explanations of the individual steps apply analogously to the apparatus components and vice versa. For example, explanations on algae separation in step 3b. of the method apply analogously to the algae removal unit of the apparatus according to the invention, and vice versa.

“Algae” in the sense of the invention are microalgae, such as Chlorella vulgaris, and also cyanobacteria that carry out photosynthesis, such as Arthrospira platensis.

The removal of a portion of the nutrient medium in step 3a. can take place in the manner of a discharge of the removed medium from the reactor, or also only by branching off within the reactor in the manner of a bypass, wherein the algae separation would then take place in this reactor bypass.

### Regarding the Reactor:

Various reactor types, such as photobioreactors, plate reactors or open basins (pond reactors) can be used as the reactor.

The inoculation of the nutrient medium according to the invention (step 1.) and the mixing (step 2.) can be performed either continuously already in the reactor or discontinuously in batch operation/Satzbetrieb mode. In the case of batch operation mode, the skilled person knows that the nutrient medium with the algae with the constant turbidity must then be transferred to the reactor according to the invention for step 3. Due to the existing risk of contamination, preferably at least step 2. is already carried out inside the reactor (of step 3.), particularly preferably also step 1.

According to the invention, steps 3a, 3b and 3c take place continuously, whereby “continuous operation” in the sense of the invention also includes short breaks of a maximum of 30 minutes in steps 3a to 3c—as long as the biomass concentration in the nutrient medium is kept constant, as according to the invention. In any case, it is also essential that a portion of the nutrient medium removed (in step 3a.) is recirculated after separation of the algae and (as step 3c.) is at least partially returned to the reactor, which corresponds to continuous operation.

Finally, it is also an object of the invention to use the apparatus according to the invention for continuous production of algae in turbidostat operation mode in a nutrient medium, in particular its use in the method according to the invention.

The articles “a” and “an” as used in this application should generally be construed to mean “one or more” unless specified otherwise or clear from context to be directed to a singular form.

An advantage of the invention is that it enables operation on an industrial scale, i.e. with a volume of >100 L. There is no previously observable maximum limit to the cultivation period with regard to the growth of the algae. Only maintenance work limits the duration of continuous algae production.

The volumetric productivity of the algae is higher in continuous operation mode with monitoring the control variable as setpoint, compared to batch operation mode.

### BRIEF DESCRIPTION OF THE DRAWINGS

FIG. 1 shows a sketch-like embodiment of the invention in which the turbidity sensor has been attached to the outside of the reactor, whereby the reactor is sensibly made of a transparent material, such as glass, and is designed as a tubular reactor.

FIG. 2 shows the results of continuous operation of a 0.5 L (laboratory) photobioreactor.

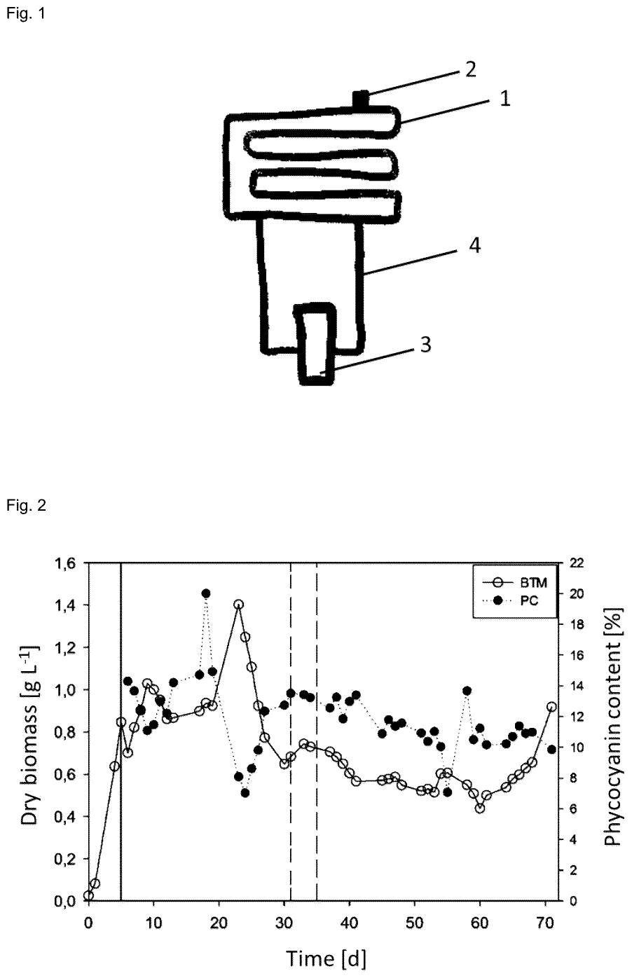

### DETAILED DESCRIPTION

In a preferred embodiment of the method according to the invention, the mixing in step 2. is carried out until a constant turbidity value of 0.4-0.9 optical density at a wavelength of the irradiated light of 750 nm is reached in the nutrient medium and this turbidity value is kept constant within this range during continuous operation in step 3. A constant turbidity value of 0.5-0.9 optical density is particularly preferred.

This type of continuous operation while maintaining the constant turbidity value in the nutrient medium is called Turbidostat operation. “Constant turbidity value” means that it does not fluctuate by more than +0.2 optical density at 750 nm around a value (within a reasonable period of 5 minutes maximum). In particular, the constant turbidity value is even within a range of +10%.

### Regarding Turbidity:

The turbidity sensor can preferably be a JUMO ecoline NTU with a measuring range between 400 and 1500 NTU. It is attached to the reactor in a way that the turbidity of the liquid in the reactor can be measured with it.

Turbidity is measured in accordance with DIN EN ISO 7027. Turbidity is preferably monitored at 750 nm (±50 nm). In any case, it has been found that turbidity measurement at this wavelength of 750 nm (±50 nm) is particularly suitable for mapping the actual algae concentration, regardless of the nutrient medium used or its usual ingredients. This is because light irradiated at 750 nm (±50 nm) is not absorbed by the algae but reflected, so that turbidity measurement is possible without error. The error in determining the actual algae concentration via the turbidity in the reactor is very small. As a result, the volumetric productivity of the algae is more constant and better reproducible, which is important for large-scale production. The turbidity sensor and its measured values are converted to optical density or biomass concentration values using calibration curves, depending on the type of algae used.

The turbidity sensor can be installed on the outside or inside of the reactor. If it is mounted on the outside, the reactor is sensibly made of a transparent material so that the turbidity sensor can detect the turbidity inside the reactor. However, as it is known that light must be irradiated during algae production, the reactor material is glass, at least in some areas of the reactor, so that the turbidity sensor can be mounted on the outside.

In a preferred embodiment of the method, the continuous operation in step 3. is carried out for at least 3 days without interruption.

In another preferred embodiment of the method according to the invention, light whose wavelength spectrum is ≥12% at 400-500 nm and ≥60% at 600-700 nm is irradiated in step 3 when regulating the control variable of the irradiated light quantity. The advantage of this is that there is little photoinhibition.

In a preferred embodiment of the method, the dilution factor and the irradiated light quantity and the temperature are regulated as control variables in step 3. Accordingly, the apparatus according to the invention in such an embodiment would also have a pump, a light source and a heating/cooling unit as regulating units.

In a preferred embodiment of the invention, the algae separation (or, correspondingly, the algae removal unit) comprises an electrocoagulation (or, correspondingly, an electrocoagulation unit) for concentrating the algae.

“Electrocoagulation” in the sense of the invention means that the algae in the nutrient medium pass through an electric field (within the electrocoagulation unit) and, due to their surface tension reduction, agglomerates into larger particles and sink to the bottom (sediment) or even rise to the surface (depending on the voltage due to decomposition of water and formation of microbubbles of H₂ and O₂). The skilled person is familiar with methods for separating the algae particles floating on top or sedimenting at the bottom from the liquid.

This means that electrocoagulation is used for flotation, sedimentation or simply for particle enlargement. Subsequent separation takes place using known methods, for example filtration (such as cross-flow filtration or rotating disc filtration, vibrating sieve, inclined filter) or hydrocyclones. “Hydrocyclones” are centrifugal separators for liquid mixtures. Hydrocyclones are used to separate or classify solid particles (such as algae) contained in suspensions. Emulsions, such as oil-water mixtures, can also be separated. In the invention, for example, algae can be separated as a separate stream, for example via a screw in the hydrocyclone.

It makes sense to pass the algae particles within the removed portion of the nutrient medium (see step 3a.) through the electrocoagulation unit in such a way that each algae particle is in the electric field of the electrocoagulation unit for at least 0.2 min.

The choice of electrocoagulation for the separation of algae (also known as harvesting or downstream) has the advantage that no interruptions are necessary for cleaning or draining. This is because either the algae clump together through this and a subsequent filter no longer clogs as much (due to the larger particle sizes of the algae). Or the algae sediment and can be removed covered by liquid, e.g. using a slider. With targeted hydrolysis of the water with bubble formation, however, it is also possible to cause flotation so that the algae rise to the top with the bubbles and can be removed there without interruption.

This embodiment thus enables shorter processing times, as maintenance (for example due to clogged filters) can be dispensed with. It is therefore particularly preferable that the algae separation comprises both electrocoagulation (unit) and a filtration (unit) or a hydrocyclone.

In any case, advantageously, electrocoagulation results in a continuous pre-concentration due to flocculation in accordance with the surface tension. This means that during subsequent separation there is considerably less clogging of the filter, for example, or a separation using a hydrocyclone is considerably easier and more effective.

This means, advantageously, that through electrocoagulation in the separating step coarser-pored membranes, sieves or filters can be used (with pore sizes of 50-100 μm, for example). This significantly reduces the maintenance effort.

In the case of a hydrocyclone, higher flow rates are advantageously possible than would be the case without electrocoagulation.

In summary, this increases the throughput in algae separation, i.e. in the so-called downstream section.

The design is correspondingly simple and therefore permits cost-effective production of the algae also on an industrial scale.

As already described, the algae to be separated are larger due to electrocoagulation and there is less maintenance (less filter clogging) or easier separation. In any case, the apparature will need to be opened less often or even not at all (during production), which significantly reduces the risk of contamination. The algae are thus favourably suited for consumption.

In a preferred embodiment of the method according to the invention, the control variable which is regulated in the reactor in step 3, is
a dilution factor of 0.15-1.0 per day and/or
an irradiated light quantity of 100-400 μmol/m² s and/or
a temperature of 15-40° C.

Regarding the dilution factor 0.28-1.0 per day (or even 0.28-0.30 per day) and regarding the temperature 15-34° C. are particularly favoured.

The advantage is that there is little photoinhibition. This is the best operation state in terms of energy. Multi-dimensional regulation using machine learning algorithms is possible.

In a preferred embodiment of the invention, the reactor is a tubular reactor (e.g. of modular design) with >6,000 L cultivation volume, in particular even 8,000 to >10,000 L.

In a preferred embodiment of the invention, a light source is comprised as a controllable lighting system, which can be controlled automatically or manually regarding intensity (settings: on, off or brighter or darker) as sole lighting or in addition to the solar irradiation. Particularly preferably, the light quantity can be measured via a PAR sensor.

In a preferred embodiment of the invention, the algae to be produced are “Limnospira maxima”.

In one embodiment of the invention, the reactor is an open basin (i.e. an open pond) or a tubular reactor.

In a preferred embodiment, the reactor is a tubular reactor. This has a particularly preferred meandering shape with a maximum of 7×35 loops arranged in three dimensions (loops as shown, for example, in EP 2 486 790 A1). The advantage of this design is that the tubular unit formed from loops fits into a standard commercial container. 6×30 loops are particularly favoured. Easy parallelization of such tubular units formed from loops is possible, container by container.

In a preferred embodiment, the reactor is a tubular reactor and the electrocoagulation unit is a section of the tubular reactor, such as a bypass, i.e. a branch of the tubular reactor, which is designed for electrocoagulation.

In a preferred embodiment of the invention, the electrocoagulation unit is a tubular unit. In a particularly preferred embodiment, the length of the electrocoagulation unit is 50 cm (±5 cm).

In a preferred embodiment of the invention, the removal unit comprises
an electrocoagulation unit within the reactor for agglomeration of the algae, and
a separation unit selected from a filtration unit and a hydrocyclone,
wherein the pipe coming from the reactor in the direction of algae flow leads first to the electrocoagulation unit and then to the separation unit (filter or hydrocyclone). In the process according to the invention, the algae separation in step 3b. by means of electrocoagulation would then be followed by a step of separating the algae by means of filtration (by means of a filter) or hydrocyclone.

Particularly preferably, the separation takes place in the separation unit (as part of the separating step/the removal unit) by means of a filter, whereby this has a pore size in the range of 2-100 μm (50-100 μm is also possible). This pore size is particularly advantageous for the invention, when the particle size of the algae is increased by means of electrocoagulation.

In a further preferred embodiment of the invention, the reactor is a tubular reactor and the removal unit (of the apparatus according to the invention) comprises:
an electrocoagulation unit within the tubular reactor (i.e. the tubular system of the reactor) for agglomeration of the algae, and
a separation unit selected from a filtration unit and a hydrocyclone,
wherein the pipe coming from the reactor in the direction of algae flow leads first to the electrocoagulation unit and then to the separation unit (filter or hydrocyclone), and
wherein the algae removal unit is designed as a closed environment so that the algae can be separated in the absence of air.

This corresponds recognisably to the embodiment of the method according to the invention, in which the reactor is a tubular reactor, and
the algae separation in step 3b. takes place by means of electrocoagulation within the tubular reactor for agglomeration with subsequent separation by means of a filter or hydrocyclone, and
wherein both the electrocoagulation and the subsequent separation take place in a closed environment (optionally with exclusion of air)—i.e. the entire algae separation in the algae removal unit according to the invention takes place in a closed environment.

The advantage of these two similar embodiments is that the design is very compact and simple.

This is because with a larger particle size (due to agglomeration during electrocoagulation), the algae particles cannot penetrate as far into the filter membrane pores and clog them. In the hydrocyclone, separation from the liquid by means of centrifugal force is possible much more precisely with a larger particle size of the algae particles to be separated. Larger filter membranes can be used or the flow speed with hydrocyclones can be increased.

The apparatus in these two embodiments requires fewer valves, branches and other sources of problems. In addition, the algae are protected from contaminants due to the closed environment and, hence, are suitable for consumption. This is because there can be no contamination of the algae produced. In the case of particularly sensitive algae species, it would also be possible to separate the algae in a protective gas atmosphere so that they are protected from unwanted oxidation. In any case, the algae are suitable for consumption, as an advantage.

It should also be comprised that the separation unit of the algae removal unit has both a filter and a hydrocyclone. It is useful to have a pipe between the two, which transports the nutrient medium from one component to the other.

In these embodiments with a closed environment, gassing with sterile filtered air is particularly preferable, preferably by using a 0.2 μm sterile filter. Depending on the algae species, it can be enriched with CO₂. Correspondingly, an apparatus would have an appropriate valve for mixing air and CO₂.

In a variant of these two embodiments with a separation unit selected from filter, this filter can be a gravity filter, a drum filter or a vibrating sieve.

In a preferred embodiment, the artificially generated light is produced by LEDs in a photosynthetically active spectrum that can be absorbed by the algae, which can be switched on or off automatically or manually according to the regulation instructions, or switched brighter or darker in intensity, respectively. This can also relate to individual spectral ranges of the LED spectrum. The advantage of LEDs is that they are energy-saving.

In an also preferred embodiment of the invention, the apparatus according to the invention comprises a regulator, a control unit and a valve, for adjusting the amount of the nutrient medium to be removed from the reactor, so that a turbidity value (in the reactor in the nutrient medium) can be maintained as a sepoint.

In an also preferred embodiment of the invention, the cathode of the electrocoagulation (or the electrocoagulation unit) is made of stainless steel and/or the anode is made of Mg, Fe or graphite. The anode is particularly preferably made of magnesium or graphite.

In an also preferred embodiment, the electrocoagulation unit comprises a tube as the cathode, in particular a stainless steel tube section, and an anode rod, in particular an anode rod made of magnesium. However, the anode rod can also be made of graphite as an inert anode.

In a particularly preferred embodiment of the invention, the cathode of the electrocoagulation unit (or of the electrocoagulation) is made of stainless steel and the anode is a sacrificial anode made of Mg. Advantageously, the algae that can be produced with the invention are thus suitable for consumption. This is because with this sacrificial anode ions are released and the algae particles sediment and remain protected from contamination and oxidation under the liquid.

Preferably, in one embodiment the electrocoagulation unit comprises a plastic protective tube which encloses both the anode and the cathode.

In an embodiment in which fresh nutrient medium is also additionally added to the reactor in step 3c. (in continuous operation mode), it is meaningful when a fresh medium pipe leads from a nutrient medium storage container (for fresh nutrient medium) to the pipe according to the invention, from the algae removal unit back to the reactor, i.e. such a fresh medium pipe is also comprised.

Preferably, in the method all media streams in all method steps are run in a closed system or are suitably protected against the entry of external soiling and contamination.

In a preferred embodiment of the method according to the invention, the constant turbidity value in step 2. is 0.5-0.9 optical density at a wavelength of the irradiated light of 750 nm.

This corresponds to an (algae-) dry biomass of 0.5-0.7 g/L in relation to the volume of the nutrient medium, for example in the case of the alga Spirulina limnospira maxima. Advantageously, this enables a more effective process, i.e. the volume-time yield of the process is very high due to the high volumetric productivity of the algae in this very narrow range.

In addition, it came out that the high volumetric productivity occurs particularly at this constant algae concentration of 0.5-0.7 g/L, whereby this range can be easily adjusted and continuously monitored via a turbidity of 0.5-0.9 optical density at 750 nm wavelength of light.

It is particularly preferred, when the constant turbidity value is even 0.6-0.8 optical density at 750 nm.

In one embodiment of the method according to the invention, it is possible that in step 3a. the amount of nutrient medium continuously removed (i.e. the continuous removal of a portion of the nutrient medium) is 8-12 vol-% per h (very preferably 10 vol-%/h), based on the total volume of nutrient medium in the reactor. Particularly preferably, the constant turbidity value is then 0.5-0.9 optical density at 750 nm. The advantage of this combination is that a maximum of the volumetric productivity of the algae (i.e. a maximum of algae growth) is achieved in the range of this (limit-) turbidity value of 0.5-0.9 optical density and that simultaneously a constant nutrient concentration is achieved with this amount of continuously exchanged medium, i.e. that in addition to the turbidostat operation also a chemostat operation mode can be present.

Particularly preferably, the entire portion of the nutrient medium (in step 3b.) is freed of algae and completely returned to the reactor (in step 3c.) without adding fresh nutrient medium. Hereby, the reprocessed nutrient medium is disinfected from contaminants by suitable treatment steps (known to the skilled person, e.g. ultrafiltration, UV treatment).

In any case, it makes sense for the volume of removed medium and the volume of added medium to be approximately the same, so that the fill level of the reactor remains largely constant. Because this topic must be separated from the question of whether the nutrient medium added in step 3c is exclusively reprocessed nutrient medium or also fresh nutrient medium.

In a preferred embodiment of the method according to the invention, the reactor is a tubular reactor and the flow speed through the tubular reactor in step 3 is >0.3 to 0.8 m/s, or even >0.5 to 0.8 m/s. This is advantageous in order to be able also to operate the electrocoagulation effectively while keeping this flow speed constant.

In an also preferred embodiment of the method according to the invention, the constant turbidity value according to the invention is 0.6-0.8 optical density at 750 nm, and the concentration of nutrients in the nutrient medium used in step 1. is at least 6 g/L NaHCO₃ and 0.23 g/L NaNO₃±15%. Very preferably, at this constant turbidity value, the NaHCO₃ is contained in the nutrient medium at 6.8-9.2 g/L (or also 8 g/L) and/or the NaNO₃ is contained at 0.23 g/L.

In a further preferred embodiment of the method according to the invention, the algae separation in step 3b. takes place via electrocoagulation in a tube section of an electrocoagulation unit with 40-60 cm in length (of the tube section) and with a flow speed (within the electrocoagulation unit) of 0.007-0.03 m/s. Advantageously, these parameters are particularly suitable for effective agglomeration via electrocoagulation for various large-scale designed variants of the invention. The tube section has a particularly preferred inner diameter of the tubular cathode of 50 mm+2 mm, with the rod-shaped anode having an outer diameter of 21 mm+2 mm.

In an also preferred embodiment, the algae separation in step 3b. takes place by means of electrocoagulation at a voltage of 12-36 V (particularly preferably 12-18 V) and with a sacrificial anode made of magnesium or an inert anode made of graphite. Advantageously, microbubble formation does not already occur (due to electrolysis of water) and yet electrocoagulation can be carried out effectively enough. This means that Algae do not rise to the top and can be separated (covered by liquid) without the risk of contamination at the bottom of the reactor or the electrocoagulation unit, respectively. Preferably, it is a tubular electrocoagulation unit, with the anode as a rod having an outer diameter of 21 mm and the cathode as a tube having an inner diameter of 50 mm.

The temperature during cultivation (especially in continuous operation in step 3.) is preferably 30° C.±2° C. Advantageously, in this case the growth rate is in a favourable range.

In a preferred embodiment of the method with a photobioreactor as the reactor, the following parameters are present:
Dilution factor (flow rate through the photobioreactor): 0.34 d-1, and/or
Concentration of dry biomass: 0.755 g L-1, and/or
Phycocyanin content: 13%, and/or
Productivity: 0.26 g L-1 d-1,
each in range limits of +10% of the respective value.

For the realisation of the invention, it is also expedient to combine the above-described variants of the invention, embodiments and features of the claims with one another. In the following, the invention will be explained in more detail by means of an embodiment example, without being limited to the features.

### Execution Example 1

The cultivation of the algae species Limnospira maxima is started in Satzbetrieb mode (=batch mode) until a constant turbidity value (here turbidity value of 0.5-0.9 optical density at 750 nm) is reached.

For turbidity measurement according to DIN EN ISO 7027:

The turbidity sensor 2 is a JUMO ecoline NTU with a measuring range between 400 and 1500 NTU. The measuring principle of the turbidity sensor 2 JUMO ecoLine NTU is based on infrared light measurement according to the 90° scattered light method.

Reactor 1 is a tubular reactor made of loops of a tube as used in EP 2 486 790 A1. Here, 6 loops were arranged next to each other in the horizontal direction and 30 loops were arranged on top of each other in the vertical direction.

The growth maximum, i.e. the maximum volumetric productivity of the algae, was set at approx. 0.6 and 0.8 optical density at 750 nm. This corresponds to a dry biomass of around 0.5-0.7 g/L.

Gassing takes place with sterile filtered air that has been temporarily enriched with CO₂.

The nutrient concentration in continuous operation mode in the reactor is: NaHCO₃ 8 g/L; NaNO₃ 230 mg/L; NPK fertiliser 0.5 mL/L.

This is followed by the transition to continuous operation, in which recycled (=nutrient medium freed from algae) and, if necessary, also fresh medium is added and algae suspension is removed.

### For Electrocoagulation:

The separation is carried out by means of electrocoagulation in an electrocoagulation unit 3 with a length of approx. 500 mm; flow speed 0.007-0.03 m/s; voltage approx. 12-18 V; minimum residence time of individual algae in the electric field of the electrocoagulation unit 0.2 min.

The electric module of the electrocoagulation unit 3 uses standard anodes from heating boiler construction and electroplating. The algae were then filtered using a 50 μm pore filter and dried.

### Execution Example 2

A 0.5 L photobioreactor system was operated in continuous mode for the strain A. platensis CCALA (T=30° C., initial photon flux density I₀=150 μmol m⁻² s⁻¹). During course of the process, the flow rate was varied in the range of 0.1-0.34 d⁻¹. The results of the biomass production (BTM) and the cellular phycocyanin content (PC) are shown in FIG. 2.

The optimum operating point of the continuous system based on biomass formation (quantity of biomass formation) and a maximum phycocyanin content (quality of biomass) is marked in FIG. 2. The operating point is characterised by the following configuration:
Flow rate: 0.34 d⁻¹
Concentration of dry biomass: 0.755 g L⁻¹
Phycocyanin content: 13%
Productivity: 0.26 g L⁻¹ d⁻¹.

### Execution Example 3: Comparison with Batch Operation

For the performance comparison between batch and continuous operation mode of the algae apparatus, both operating modes were realised in the algae apparatus. Based on the experimental data, the comparison of the process parameters from 100 L-10,000 L scale is shown in the following tables.

Calculation 1 week - volume 100 L

| Continuously | | Reference (batch) |
|---|---|---|
| Duration [d] | | 7 |
| Dry mass [kg] | 0.00059 | 0.0014 |
| Dilution factor D [d]⁻¹ | 0.29 | 0 |
| Start volume [L] | 100.00 | 100 |
| Throughput per day [L] | 29.00 | 0 |
| Throughput during the period [L] | 203.00 | 0 |
| Total volume [L] | 303.00 | 100 |

| | | |
|---|---|---|
| Biomass produced [kg] | 0.18 | 0.13 |
| Percentage increase compared to reference [%] | 30.08 | |

Volume 10,000 L

| Continuously | | Reference (batch) |
|---|---|---|
| Duration [d] | | 7 |
| Dry mass [kg] | 0.00059 | 0.00125 |
| Dilution factor D [d]⁻¹ | 0.29 | 0 |
| Start volume [L] | 10000.00 | 10000 |
| Throughput per day [L] | 2900.00 | 0 |
| Throughput during the period [L] | 20300.00 | 0 |
| Total volume | 30300.00 | 10000 |

| | | |
|---|---|---|
| Biomass produced [kg] | 17.88 | 12.50 |
| Percentage increase compared to reference [%] | 30.08 | |

Calculation 1 month - volume 100 L

| Continuously | | Reference (batch) |
|---|---|---|
| Duration [d] | 30 | 26 |
| Dry mass [kg] | 0.00059 | 0.00125 |
| Dilution factor D [d]⁻¹ | 0.29 | 0 |
| Start volume [L] | 100.00 | 100 |
| Throughput per day [L] | 29.00 | 0 |
| Throughput during the period [L] | 870.00 | 0 |
| Total volume [L] | 970.00 | 240 |

| | | |
|---|---|---|
| Biomass produced [kg] | 0.57 | 0.28 |
| Percentage increase compared to reference [%] | 207.36 | |

Volume 10,000 L

| Continuously | | Reference (batch) |
|---|---|---|
| Duration [d] | 30 | 26 |
| Dry mass [kg] | 0.00059 | 0.00125 |
| Dilution factor D [d]⁻¹ | 0.29 | 0 |
| Start volume [L] | 6000.00 | 10000 |
| Throughput per day [L] | 1740.00 | 0 |
| Throughput during the period [L] | 52200.00 | 0 |
| Total volume | 58200.00 | 24000 |
| Biomass produced [kg] | 57.23 | 27.60 |
| Percentage increase compared to | 207.36 | |
| reference [%] | | |

The results show that continuous operation achieves a 30% increase in biomass productivity already after one week of operation. With continuous operation over a month, biomass production can be roughly doubled. The following assumptions were made for the calculation:
A maximum of three batch modes/Satzbetriebe per month due to intermediate harvesting and cleaning cycles,
For the productivity, results from at least 5 reference tests were used (batch operation) or at least 5 days in stable continuous operating mode were averaged.
In batch mode, in each case 20% of the volume must be used for new inoculation.
Continuous operation is subject to cleaning cycles during operation.

### REFERENCE SIGN

1 Reactor (with nutrient medium)
2 Turbidity sensor
3 Electrocoagulation unit (algae removal unit)
4 Pipe between reactor and algae separation unit

1. A method for continuous production of algae in a nutrient medium comprises using the apparatus according to claim 12 to perform the following steps:
1. inoculating the nutrient medium with algae;
2. mixing until a constant biomass concentration of 0.3-0.8 g/L in the nutrient medium is reached;
3. continuously operating the algae cultivation in the reactor (1) while maintaining the constant biomass concentration, comprising
3a. removing a portion of the nutrient medium, and
3b. separating the algae thereof, and
3c. adding at least a portion of the nutrient medium removed in step 3a. after algae separation as algae-free, reprocessed nutrient medium; and
4. regulating a control variable selected from a dilution factor, an irradiated light quantity, and a temperature in the reactor, while continuously operating the algae cultivation and thereby maintaining the constant biomass concentration.

2. The method according to claim 1, wherein the mixing in step 2 is carried out until a constant turbidity value of 0.4-0.9 optical density at a wavelength of irradiated light of 750 nm is reached in the nutrient medium and this turbidity value is kept constant within this range while continuous operating in step 3.

3. The method according to claim 1, wherein the continuous operating in step 3 is carried out for at least 3 days without interruption.

4. The method according to claim 1, wherein, when regulating the control variable of the irradiated light quantity in step 3, light is irradiated with a wavelength spectrum ≥12% at 400-500 nm and ≥60% at 600-700 nm.

5. The method according to claim 1, wherein the dilution factor and the irradiated light quantity and the temperature are regulated as the control variable in step 3.

6. The method according to claim 1, wherein separating the algae comprises an electrocoagulation (3) for concentration.

7. The method according to claim 1, wherein the control variable which is regulated in step 3 within the reactor, is a dilution factor of 0.15-1.0 per day, and/or
an irradiated light quantity of 100-400 μmol/m²s, and/or
is a temperature of 15-40° C.

8. The method according to claim 1, wherein the reactor (1) is a tubular reactor, and
separating the algae in step 3b takes place by electrocoagulation (3) within the tubular reactor for agglomeration with subsequent separation by filtration or hydrocyclone, and
wherein both the electrocoagulation (3) and the subsequent separation take place in a closed environment.

9. The method according to claim 1, wherein the reactor (1) is a tubular reactor and a flow rate through the tubular reactor in step 3 is >0.3 to 0.8 m/s.

10. The method according to claim 1, wherein separating the algae in step 3b takes place by electrocoagulation in a tube section of an electrocoagulation unit (3) of 40-60 cm length and with a flow rate within the electrocoagulation unit of 0.007-0.03 m/s.

11. The method according to claim 1, wherein separating the algae in step 3b takes place by electrocoagulation at a voltage of 12-36 V and with a sacrificial anode of magnesium or an inert anode of graphite.

12. An apparatus for continuous production of algae in a nutrient medium, comprising:
a reactor (1);
a turbidity sensor (2) connected to the reactor for monitoring the turbidity of the nutrient medium within the reactor;
an algae removal unit;
a pipe (4) from the reactor to the algae removal unit and from there back to the reactor; and
a regulation unit, for regulating a control variable inside the reactor, including
a pump for regulating a dilution factor, and/or
a light source for regulating a light quantity, and/or
a heating/cooling unit for regulating a temperature,
each with a control unit for synchronising the turbidity sensor with the regulation unit so that a turbidity value can be maintained as a setpoint.

13. The apparatus according to claim 12, wherein the algae removal unit comprises an electrocoagulation unit (3).

14. The apparatus according to claim 12, wherein the reactor (1) is a tubular reactor and the removal unit comprises an
electrocoagulation unit (3) within the tubular reactor for agglomeration of the algae, and
a separation unit selected from a filtration unit and a hydrocyclone, and
wherein the pipe (4) coming from the tubular reactor in a direction of algae flow leads first to the electrocoagulation unit (3) and then to a filter or hydrocyclone, and
wherein the algae removal unit is designed as a closed environment so that the algae can be separated in the absence of air.
